(19) United States
(12) Patent Application Publication
(10) Pub. No.: US 2014/0005582 A1
(43) Pub. Date: Jan. 2, 2014

(54) Leg cast covering apparatus

(71) Applicants: Tim D. Dickison, Santa Monica, CA (US); Harvey A. Rosenthal, Santa Monica, CA (US)

(72) Inventors: Tim D. Dickison, Santa Monica, CA (US); Harvey A. Rosenthal, Santa Monica, CA (US)

(21) Appl. No.: 13/507,465

(22) Filed: Jul. 2, 2012

(51) Int. Cl. A61F 5/00

(52) U.S. Cl. 602/3

(57) ABSTRACT

A cast covering apparatus comprising a substantially tubular construction adapted to be disposed about an orthopedic leg cast on the user's leg and foot. The covering is constructed of an elastic textile having a smooth inner surface that prevents entanglement with typical surface projections of an orthopedic cast. At the end portion of the covering adapted to be disposed adjacent the user's toes, the covering extends into a toe opening and lower and upper toe panels. The lower toe panel extends across the sole of the user's foot. The upper toe panel extends over the instep of the user's foot, the opposed ends of the upper panel being sewed to the sides of the tubing. When the upper toe panel is adjacent the lower toe panel, the user's toes will be covered and when the upper toe panel is folded back along the user's instep, the toes are exposed.

### BACKGROUND OF THE INVENTION

1. Field of the Invention

The present invention generally relates to covering apparatus for leg, ankle and foot casts and specifically to those covers that permit the user's toes to be covered or exposed.

2. Prior Art

The present invention is intended to provide an improved orthopedic cast cover which envelops the user's lower leg and foot and the orthopedic cast and alternatively allows the user's toes to be exposed or covered. One of the devices taught by the prior art employs complementing foot and leg covers which overlap when applied to jointly cover the cast and foot. The portion of the invention used to cover the user's foot has an elastic ankle strap which retains it on the foot and the leg sock has a heel portion adjoining the foot opening. The problem inherent in this device stems from the requirement for cooperation between the foot and leg covers. Since the portion of the cover intended to be about the user's foot is held in place only by an elastic band, the two complementary elements can be displaced from one another.

Another device taught by the prior art is a toe covering for use in connection with an orthopedic cast when it is applied to a user's foot or leg. The device relates to a toe covering comprising an open-ended portion adapted to receive the forward portion of the user's foot extending beyond the orthopedic cast and which, when encasing the forward portion of the user's foot, extends over the forward portion of the cast and is detachably secured thereto. The problems inherent in this design relate to the limited function of covering only the user's toes. Since the device covers only the user's toe, it has no function to prevent the rough surface of an orthopedic cast from coming in contact with the user, the user's clothing or items in close contact thereto such as home furnishings, bed sheets or any materials that could snag or become entangled with the surface of the orthopedic cast.

The present invention obviates the inadequacies inherent in those devices taught by the prior art that are employed to partially or wholly cover orthopedic casts. The present invention comprises a tubular covering constructed of an elastic textile material having a smooth inner surface that envelops the outside of the orthopedic cast preventing its contact with dirt or other damaging media while preventing entanglement with surface projections extending from an orthopedic cast. The upper tubular sleeve of the present invention fully covers the Portion of the cast adjacent the user's lower leg and ankle. An integral lower tubular sleeve of the present invention is an extension from the upper tubular sleeve and extends into a closable flap that consists of upper and lower toe panels. The lower toe panel extends across the sole of the user's foot. The upper toe panel is positioned forwardly from the instep of the foot. When the upper toe panel is folded to be adjacent to the lower toe panel, the user's toes will be covered. When the upper toe panel is folded back along the user's instep, the toes are exposed.

### SUMMARY OF THE INVENTION

The present invention orthopedic cast covering apparatus comprises a substantially tubular element that is adapted to be disposed about an orthopedic leg cast on the user's leg, ankle and foot. The invention is constructed of an elastic textile that is a combination of polyester and a synthetic fiber of exceptional elasticity, forms thereof being generally referred to as elastane or Spandex. The present invention is used to cover the full outer surface area of an orthopedic cast on the user's leg, ankle and foot. As described in detail hereinbelow, an objective of the present invention is to cover the orthopedic cast with a material that will reject entanglement with surface projections typically displayed by some orthopedic casts. To meet this objective, the synthetic fiber from which the textile is made is coated with a polymer that will withstand entanglement. The present invention cast cover includes a tubular main body comprised of an upper tubular sleeve and a contiguous lower tubular sleeve and a closable flap that is a continuation of the lower tubular sleeve and is adapted to be disposed adjacent the user's toes. The closable flap of the main body is comprised of upper and lower toe panels. The lower toe panel extends along the bottom of the lower tubular sleeve and is positioned adjacent the sole of the user's foot. The upper toe panel of the closable flap has defined opposed first and second side edges when the lower tubular sleeve and the depending closable flap are flattened. The side edges of the upper toe panel are secured to the interface between the main body of the lower tubular sleeve and the lower panel. When the upper toe panel is fully extended around the user's toes and is positioned adjacent the lower toe panel, the user's toes will be covered and protected from the environment. To expose the user's toes, the upper toe panel may be folded back along a fold line until the opposite first and second side edges of the end portion are substantially in alignment respective edges of the main body of the lower tubular sleeve.

It is therefore an object of the present invention to provide a cast covering apparatus that can fully envelop an orthopedic cast applied to the user's leg, ankle and foot.

It is another object of the present invention to provide a leg covering apparatus that will avoid entanglement or being snagged upon projections from the surface of the orthopedic leg cast.

It is still another object of the present invention to provide a convertible toe opening to expose the toes for cooling, massaging and the like without having to remove the cast cover.

It is still yet another object of the present invention to provide an improved cast covering apparatus that is simple and inexpensive to fabricate.

The novel features which are believed to be characteristic of the invention, both as to its organization and method of operation, together with further objectives and advantages thereof, will be better understood from the following description considered in connection with the accompanying drawing in which a presently preferred embodiment of the invention is illustrated by way of example. It is to be expressly understood, however, that the drawing is for the purpose of illustration and description only, and is not intended as a definition of the limits of the invention.

### BRIEF DESCRIPTION OF THE DRAWING

FIG. 1 is a perspective view illustrating the present invention leg cast covering apparatus in operative position on a user's leg having a leg, ankle and foot cast.

FIG. 2 is a bottom plan view of the present invention leg cast covering apparatus illustrating the upper toe panel in an unfolded position.

FIG. 3 is a top plan view of the leg cast covering apparatus illustrating the upper toe panel in an unfolded position.

FIG. 4 is a bottom plan view of the present invention leg cast covering apparatus illustrating the upper toe panel in a folded position adjacent the instep.

FIG. 5 is a top plan view of the leg cast covering apparatus illustrating the upper panel in an folded position adjacent the instep.

FIG. 6 is a partial, side elevation view of the leg cast covering apparatus illustrating the upper toe panel in a folded position adjacent the instep.

FIG. 7 is a partial, side elevation view of the present invention leg cast covering apparatus illustrating the upper toe panel in a closed position.

### DESCRIPTION OF THE PRESENTLY PREFERRED EMBODIMENT

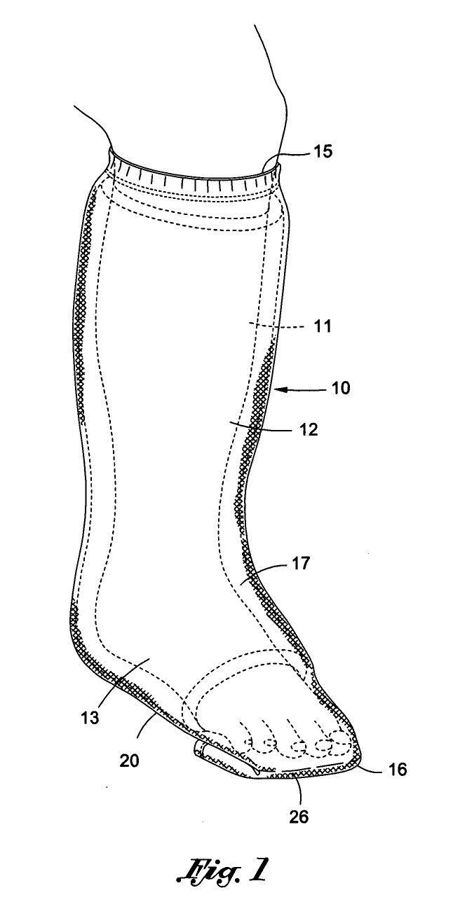

An understanding of the purpose of the present invention can be best gained by reference to FIG. 1 wherein the leg cast covering apparatus is generally referred to by the reference numeral 10. A leg cast cover 10 in accordance with the present invention comprises an apparatus to cover an orthopedic cast 11 and an injured persons leg, ankle and foot on which it is placed. It is an objective of the present invention to provide a leg cast cover that can be removably but securely placed about the leg cast 11. To meet this objective, leg covering 10 comprises an upper tubular sleeve 12 to cover the portion of leg cast 11 about the user's leg and ankle and an extended contiguous lower tubular sleeve 13 to be positioned about the lower portion of the cast and the foot of the user. The contiguous upper and lower tubular sleeves 12 and 13 are preferably constructed of an elastic textile that is a combination of polyester and a synthetic fiber of exceptional elasticity, exemplary forms of the latter generally being referred to as elastane or Spandex. Since it is another object of the present invention to provide a leg cast covering apparatus that will not snag on or be engaged with a rough portion or projection from the exterior of the leg cast 11, the synthetic fiber from which the textile is coated with a polymer that provides substantial surface tension and thereby prevents penetration of the surface of the textile material.

The upper tubular sleeve 12 of leg cast cover 10 has an open end 15 for entry through which the cast 11 can be inserted. Upper end 15 is elasticized and will maintain upper member 12 in place once the user's foot and leg cast 11 have been inserted. Upper tubular sleeve 12 provides a substantially concave entry for the user's foot and extends contiguously into lower tubular sleeve 13 which is in the form of a pocket which depends into a foldable flap 16. As will be described in detail hereinbelow, foldable flap 16 provides the means for covering the user's toes or, in the alternative, can be folded back and be substantially adjacent the user's instep 17 thereby exposing the user's toes to ambient air for cooling and the like.

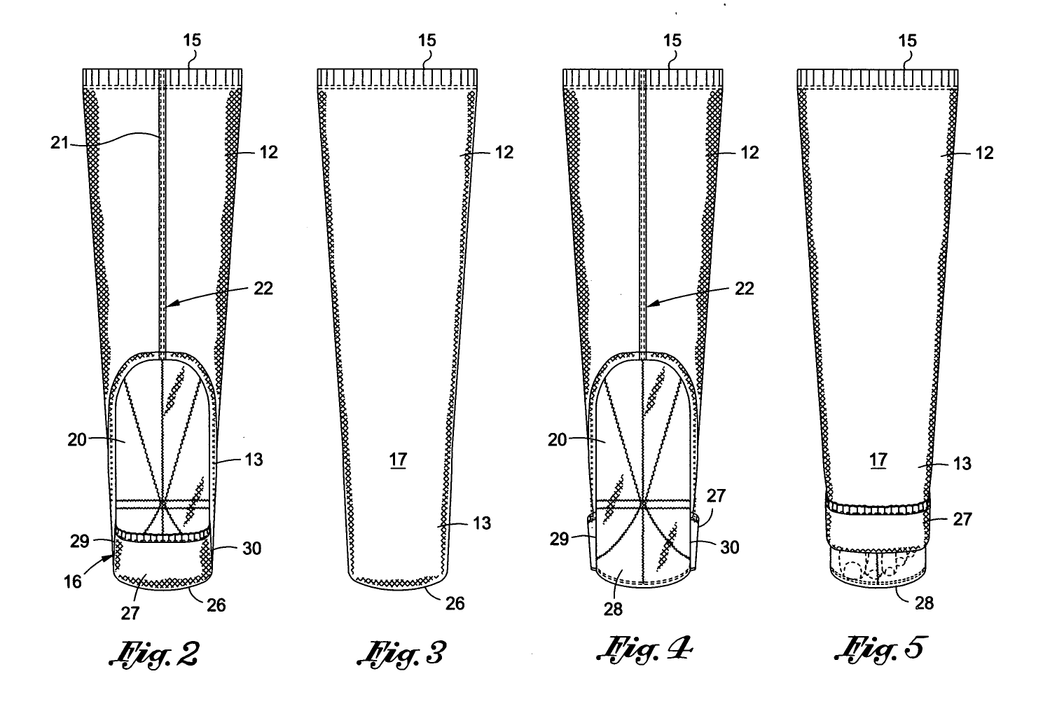

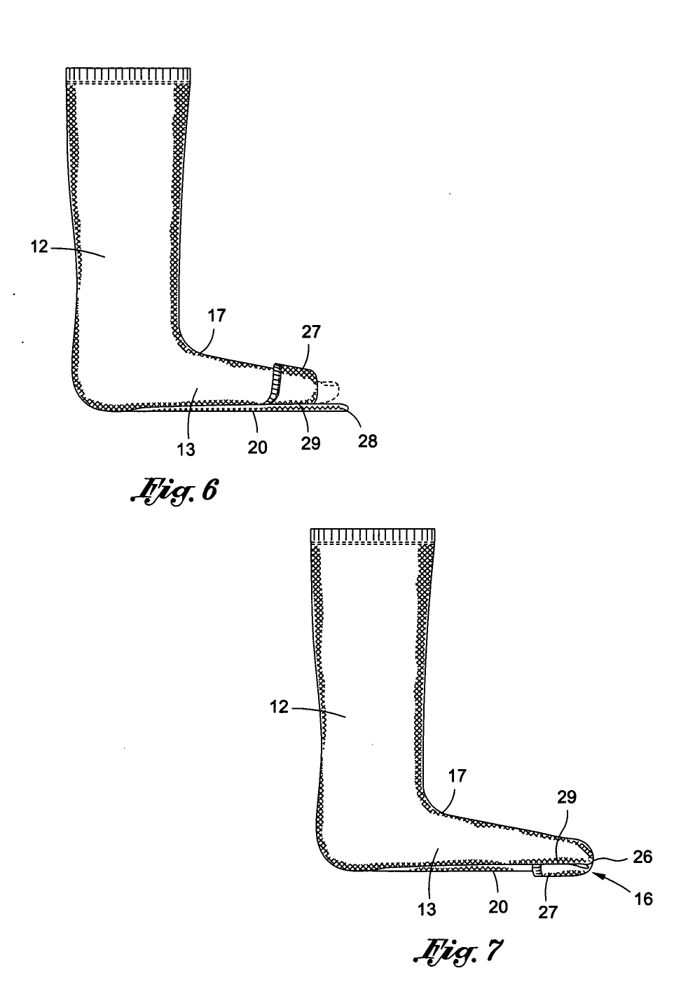

The construction of the preferred embodiment of the present invention leg cast cover 10 can be best seen by reference to FIGS. 2-7, inclusive. The present invention leg cast cover 10 is constructed from a panel of an elastic textile fabric as described. FIG. 2 illustrates the portion of the upper tubular sleeve 12 that extends along the rear of the user's leg and the portion of the lower tubular sleeve 13 that includes sole panel 20 adapted to be positioned adjacent the sole of the user's foot forwardly from the heel. The tubular structure of upper tubular sleeve 12 is formed from a panel of the elastic, textile material by securing opposing edges 21 by any suitable means, such as stitching 22, heat sealing, ultrasonic sealing or the like.

The contiguous lower tubular sleeve 13 extends forwardly into closable flap. 16. The portion of lower tubular sleeve 13 adjacent the instep 17 of the user is extended forwardly into a closable flap 16 comprising upper toe panel 27 having lateral side edges 29 and 30. Sole panel 20 is extended forwardly into lower toe panel 28. Side edges 29 and 30 of upper toe panel 27 are secured to the lateral margins of lower toe panel 28. To cover the user's toes, closable flap 16 may be folded upon itself at fold line 26 until upper toe panel 27 is positioned adjacent the margins between sole panel 20 and lower toe panel 28. The opposed lateral side edges 29 and 30 of upper toe panel 27 are secured to lower toe panel 28 by any suitable means such as sewing and the like. Since the elastic textile from which the present invention leg cast cover 10 is made is stretchable, upper toe panel 27 can be positioned adjacent instep 17 by rotating it about the interface between side edges 29 and 30 to lower toe panel 28.

The present invention provides a leg cast covering apparatus that resolves the problems inherent in those devices disclosed in the prior art. The present invention comprises a covering for a leg cast that will protect the surface of the cast from dirt or inadvertent contact with environmental contaminates. Since it is constructed of a textile fabric that is highly elastic and which is coated with a polymer that enhances surface tension, the present invention will not be entangled with or snag upon portions of the surface of the cast. Most importantly, the present invention provides an integral foldable flap that permits the toes of the user to either be covered and thereby out of contact with the ambient environment or, when necessary or desired, easily rotated to expose the user's toes to the open air.

We claim:

1. A leg cast cover for use with an orthopedic cast mounted on a user's leg, ankle and foot comprising a continuous tube of elastic textile having an upper tubular sleeve and a contiguous lower tubular sleeve, said lower tubular sleeve extending into a lower toe panel and a foldable flap having an upper toe panel in opposition to said lower tow panel, said upper tubular sleeve, lower tubular sleeve and foldable flap having opposed side edges when said upper and lower tubular sleeves and upper and lower toe panels are respectively flattened, said upper toe panel having lateral first and second side edges secured to the respective side edges of the opposed lower toe panel whereby said upper toe panel may selectively be folded back on an upper position of said lower tubular sleeve so as to be positioned to a top of the user's foot and expose the user's toes or be folded forwardly adjacent said lower toe panel and be positioned to a bottom of the user's foot covering the toes of the user.

2. A leg cast cover as defined in claim 1 wherein said elastic textile is a combination of polyester and a polymer coated elastic synthetic fiber.

3. A leg cast cover as defined in claim 2 wherein said synthetic fiber is Spandex.

4. A leg cast cover as defined in claim 1 wherein said upper tubular sleeve includes an open elasticized end whereby said upper tubular sleeve is maintained in position after the insertion of the user's leg, ankle and foot.

5. A leg cast cover as defined in claim 1 wherein said upper toe panel is elasticized whereby it is maintained in a fixed position when folded back or folded forwardly.

6. A leg cast cover for use with an orthopedic cast mounted on a user's leg, ankle and foot comprising a continuous use of elastic textile and includes an upper tubular sleeve having an elasticized open end and a contiguous lower tubular sleeve, said lower tubular sleeve extending into a lower toe panel and a foldable flap having an elasticized upper toe panel in opposition to said lower toe panel, said upper tubular sleeve, lower tubular sleeve and foldable flap having opposed side edges when said upper and lower tubular sleeves and upper and lower toe panels are respectively flattened, said elasticized upper toe panel having lateral first and second side edges secured to the respective side edges of the opposed lower toe panel whereby said elasticized upper toe panel may selectively be folded back on an upper position of said lower tubular sleeve or be folded forwardly adjacent said lower toe panel whereby the user's toes would be exposed or covered respectively.

7. A leg cast cover as defined in claim 6 wherein said elastic textile is a combination of polyester and a polymer coated elastic synthetic fiber.

8. A leg cast cover as defined in claim 7 wherein said synthetic fiber is Spandex.
